(19) United States
(12) Patent Application Publication
(10) Pub. No.: US 2025/0302723 A1
(43) Pub. Date: Oct. 2, 2025

(54) LONG-WEAR COMPOSITIONS

(71) Applicant: L'OREAL, PARIS (FR)

(72) Inventors: Jody EBANKS, CLARK, NJ (US); Tsang-Min HUANG, SCOTCH PLAINS, NJ (US)

(73) Assignee: L'OREAL, PARIS (FR)

(21) Appl. No.: 18/618,229

(22) Filed: Mar. 27, 2024

(51) Int. Cl. A61K 8/73; A61K 8/892; A61Q 1/04

(52) U.S. Cl. CPC A61K 8/73; A61K 8/892; A61Q 1/04; A61K 2800/31; A61K 2800/95

(57) ABSTRACT

Compositions including (A) at least one copolymer containing a silicone resin segment and at least one fluid silicone segment and (B) at least one hydrophobically-modified pullulan compound, are provided. Methods for making up the lips using these compositions are also provided.

### FIELD OF THE INVENTION

The present invention relates to long-wear compositions comprising (1) at least one copolymer containing a silicone resin segment and at least one fluid silicone segment and (2) at least one hydrophobically-modified pullulan compound.

### DISCUSSION OF THE BACKGROUND

Many cosmetic compositions, including pigmented cosmetics such as foundations, mascaras and lipsticks, have been formulated in an attempt to possess long wearing properties upon application. Unfortunately, many of these compositions do not generally possess good long-wear/transfer-resistance properties as well as good application properties, good comfort properties and/or good appearance or stability properties (for example, consistency or uniformity in color over time).

For example, with respect to lip compositions, commercial products containing silicon resins such as MQ resins are known. Such products are known to provide good long wear properties and/or transfer-resistance. However, such products possess poor application properties and/or poor feel upon application (for example, feels rough). Sometimes a second composition (topcoat) is separately applied to such products to improve poor properties of the compositions to make the products acceptable to consumers. Furthermore, the topcoat composition must be reapplied continually so that the product remains acceptable to consumers, meaning that the products are effectively not “long-wearing” as they require constant maintenance and reapplication. Indeed, even with a separate topcoat composition it is still difficult to achieve superior properties.

There remains a need for improved compositions having improved cosmetic properties, particularly good properties such as minimal or no flaking or cracking, good oil- and/or water-resistance, and/or good transfer-resistance properties after application.

Accordingly, one aspect of the present invention is a care and/or makeup and/or treatment composition for keratinous materials which has one or more of such properties noted above after application.

### SUMMARY OF THE INVENTION

The present invention relates to compositions including (1) at least one copolymer containing a silicone resin segment and at least one fluid silicone segment and (2) at least one hydrophobically-modified pullulan compound.

According to certain embodiments, the (A) at least one copolymer containing a silicone resin segment and at least one fluid silicone segment and the (B) at least one hydrophobically-modified pullulan compound are present in a ratio by weight, A:B, that is greater than 1:1, such as greater than about 2:1, such as greater than about 3:1, such as greater than 1:1 to about 10:1. According to certain embodiments, the (A) at least one copolymer containing a silicone resin segment and at least one fluid silicone segment and the (B) at least one hydrophobically-modified pullulan compound collectively represent a majority fraction of a film-forming component of the composition.

According to certain other embodiments, the composition is anhydrous. According to certain other embodiments the composition is liquid. According to certain other embodiments, the composition further includes at least one coloring agent. According to certain other embodiments, the composition further includes at least one volatile oil. According to yet other embodiments, the composition includes from about 10% to about 60% by weight of at least one volatile oil and wherein the (A) at least one copolymer containing a silicone resin segment and at least one fluid silicone segment and (B) at least one hydrophobically-modified pullulan compound are present in a total percent by weight from about 2% to about 20%, wherein all percentages are of the total composition.

The composition, may, in yet other embodiments meet one, a plurality, or even all of these above-described limitations.

According to another aspect, a method of making up lips includes applying any of the above-described compositions to lips in an amount sufficient to makeup the lips.

It is to be understood that both the foregoing general description and the following detailed description are exemplary/explanatory only and that they are not restrictive of the invention.

### DETAILED DESCRIPTION OF THE INVENTION

In the following description of the invention and the claims appended hereto, it is to be understood that the terms used have their ordinary and accustomed meanings in the art, unless otherwise specified.

Other than in the operating examples, or where otherwise indicated, all numbers expressing quantities (e.g., concentrations, ratios, and the like) of ingredients and/or reaction conditions are to be understood as being modified in certain embodiments by the term “about,” meaning within 10% to 15% of the indicated number (e.g. “about 10%” means 8.5% to 11.5% such as 9% to 11%, and “about 2%” means from 1.7% to 2.3 such as from 1.8% to 2.2%).

Similarly, for ratios, the modifier “about” means within 10% or 15% of the ratio. For example, about 4:1 means from 3.4:1 to 4.6:1, preferably 3.6:1 to 4.4:1. As readily understood by one skilled in the art, where the first ingredient in a ratio is less than the second, then a ratio may be expressed “inversely.” For example, if a second ingredient, B is present in an amount or concentration that is 2.5 times greater than that of ingredient A, this may be identified as an A:B ratio of 1:2.5. “About 1:10,” means from 1:8.5 to 1:11.5, preferably 1:9 to 1:11. As one skilled in the art will understand, the modifier “about” also encompasses the exact number or ratio specified. Unless otherwise indicated, all concentrations shown as percentages are concentrations by weight and also, unless otherwise indicated, relate to the entire composition as a whole.

“A” or “an” as used herein means “at least one.”

“At least one” means one or more and thus includes individual components as well as mixtures/combinations.

Numerical ranges are inclusive of endpoints and meant to include all combinations and sub-combinations. Notably, from about 5%, 10% or 15% to about 20%, 50% or 60% may refer to about 5% to about 20%, about 5% to about 50%, about 5% to about 60%, about 10% to about 20%, about 10% to about 50%, about 10% to about 60%, about 15% to about 20%, about 15% to about 50%, or about 15% to about 60%. As used herein a range of ratios is meant to include every specific ratio within, and combination of subranges between the given ranges.

“Film former”, “film-forming polymer” or “film-forming agent” as used herein means a polymer or resin that leaves a film on the substrate to which it is applied, for example, after a solvent accompanying the film former has evaporated, absorbed into and/or dissipated on the substrate.

“Wax” as used herein is a lipophilic fatty compound that is solid at ambient temperature (25° C.) and changes from the solid to the liquid state reversibly, having a melting temperature of more than 30° C. and, for example, more than 45° C., and a hardness of more than 0.5 MPa at ambient temperature.

“Surfactant” and “emulsifier” are used interchangeably throughout this specification.

“Polymer” as used herein means a compound which is made up of at least two monomers.

“Keratinous materials” includes materials containing keratin such as hair, skin, eyebrows, lips and nails.

“Substituted” as used herein (unless otherwise indicated), means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, hydroxyalkyl groups, and polysiloxane groups. The substituent(s) may be further substituted.

“Volatile”, as used herein, means having a flash point of less than about 100° C.

“Non-volatile”, as used herein, means having a flash point of greater than about 100° C.

“Anhydrous” means the compositions contain less than 1% water. Preferably, the compositions of the present invention contain less than 0.5% water, and preferably no water (free of water).

“Transfer” as used herein refers to the displacement of a fraction of a composition which has been applied to a keratinous material by contact with another substrate, whether of the same nature or of a different nature. For example, when a composition such as a liquid lipstick has been applied, the composition can be transferred onto teeth or hands, or onto the cheek of another person. Irrespective of composition type, the composition can also transfer from the keratinous material to which it has been applied to another substrate such as napkins, collars, glasses, cups or other containers.

“Transfer-resistance” as used herein refers to the quality exhibited by a composition in resisting transfer. To determine transfer-resistance, the amount of composition transferred from a keratinous material to a substrate may be evaluated and compared. For example, a composition may be transfer-resistant if, after application to a keratinous material such as lips, skin or eyelashes and contact with a substrate, a majority of the composition is left on the wearer. Further, the amount transferred may be compared with that transferred by other compositions, such as commercially-available compositions. In a preferred embodiment of the present invention, little or no composition is transferred to the substrate from the keratinous material.

“Long wear” compositions as used herein refers to compositions where the compositions, after application to a keratinous material, do not transfer or smudge after contact with another substrate and retain a consistent appearance on the keratinous material for an extended period of time. “Long wear” compositions, as used herein can also refer to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to keratinous materials such as skin, eyelashes or lips and evaluating the color of the composition after an extended period of time. For example, the color of a composition may be evaluated immediately following application to the keratinous material and these characteristics may then be re-evaluated and compared after a certain amount of time. Further, these characteristics may be evaluated with respect to other compositions, such as commercially available compositions. Additionally, long wear properties may be evaluated by applying a sample, allowing it to dry, and then abrading the sample to determine removal/loss of sample.

The composition of the present invention may be in any form, either liquid or non-liquid (semi-solid, soft solid, solid, etc.). For example, it may be a paste, a solid, a gel, or a cream. It may be an emulsion, such as an oil-in-water or water-in-oil emulsion, a multiple emulsion, such as an oil-in-water-in-oil emulsion or a water-in-oil-in-water emulsion, or a solid, rigid or supple gel. The composition of the invention may, for example, comprise an external or continuous fatty phase.

“Free” or “substantially free” or “devoid of” as it is used herein means that while it is preferred that no amount of the specific component be present in the composition, it is possible to have very small amounts of it in the compositions of the invention provided that these amounts do not materially affect at least one, preferably most, of the advantageous properties of the compositions of the invention. Thus, for example, “free of animal-derived ingredients” means that an effective amount (that is, more than trace amounts) of animal-derived ingredient(s) is/are omitted from the composition (that is, about 0% by weight), “substantially free of animal-derived ingredients” means that animal-derived ingredient(s) is/are present in amounts not greater than 0.1% by weight, and “devoid of animal-derived ingredients” means that animal-derived ingredient(s) is/are present in amounts not greater than 0.25% by weight, based on the total weight of the composition. Also, for example, “free of surfactant” means that an effective amount of surfactant is omitted from the composition (that is, 0% by weight), “substantially free of surfactant” means that surfactant is present in amounts not greater than 0.1% by weight, and “devoid of surfactant” means that surfactant is present in amounts not greater than 0.25% by weight, based on the total weight of the composition. The same nomenclature applies for all other ingredients identified throughout the application and in this paragraph such as, for example, wax (compositions of the invention which are “free of wax,” “substantially free of wax,” and “devoid of wax” have meanings consistent with the discussion within this paragraph), even if not specifically discussed for each identified ingredient. Discussed examples of the use of such language such as those in this paragraph are intended to be exemplary, not limiting.

“Physiologically acceptable” means compatible with keratinous material and having a pleasant color, odor and feel, and which does not cause any unacceptable discomfort liable to discourage a consumer from using the composition. Acceptable pH levels for compositions of the present invention are preferably acidic, that is, less than 7, preferably 6.5 or less, preferably 6.0 or less, preferably 5.5 or less, including all ranges and subranges therebetween such as, for example 3 to 5, 4 to 6, 3 to 4.5, etc. Compositions of the present invention may be in the form of a gel composition.

“Natural compound” refers to any compound derived directly from a natural substance such as a plant without having undergone any chemical modification.

“Compound of natural origin” refers to any compound derived from a natural compound which has undergone one or more chemical modifications, for example by organic synthesis reaction, without the properties of the natural compound having been modified.

“Synthetic compound” refers to any compound which is not a natural compound or a compound of natural origin.

“Room temperature” means 25° C.

“Atmospheric pressure” means 760 mmHg, i.e. 105 pascals.

“Liquid” means a composition which can conform to a container into which it is placed.

The compositions and methods of the present invention can comprise, consist of, or consist essentially of the essential elements and limitations of the invention described herein, as well as any additional or optional ingredients, components, or limitations described herein or otherwise useful. For example, the film forming system or film forming component can consist essentially of (1) at least one copolymer containing a silicone resin segment and at least one fluid silicone segment and (2) at least one hydrophobically-modified pullulan compound. Similarly, the oil system or oil component can consist essentially of, or consist of, silicone oils (volatile or non-volatile), or volatile (silicone or hydrocarbon) oils.

For purposes of the present invention, the “basic and novel property” associated with compositions, components and methods which “consist essentially of” identified ingredients or actions is “combined long wear and flexibility.”

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. Separately and furthermore, all component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Copolymer Containing a Silicone Resin Segment and a Fluid Silicone Segment

According to the first aspect of the present invention, the composition comprises at least one copolymer containing a silicone resin segment and a fluid silicone segment, also called as “silicone copolymer” in the present application.

The silicone copolymer is derived from the reaction between a silicone resin and a fluid silicone. These copolymers are described in patent applications WO 03/026 596, WO 2004/073 626, WO 2007/051 505 and WO 2007/051 506 for various cosmetic applications on hair and nails and for pharmaceutical applications on the skin.

Such copolymers are also described, for example, in “Silicone Pressure Sensitive Adhesive”, Sobieski and Tangney, Handbook of Pressure Sensitive Adhesive Technology (D. Satas Ed.), Von Nostrand Reinhold, New York.

### Silicone Resin Segment

According to one of the embodiments of the present invention, the silicone resin segment of the copolymer is a MQ type silicone resin. Examples of such MQ type silicone resins, include, but are not limited to: (i) the alkyl siloxysilicates of formula [(R1) 3SiO ½] x (SiO 4/2) y (MQ units) in which x and y are integers ranging from 50 to 80, and the group R1 represents a hydrocarbon-based radical containing from 1 to 10 carbon atoms, a phenyl group, a phenylalkyl group or a hydroxyl group, and preferably is an alkyl group containing from 1 to 8 carbon atoms, preferably a methyl group; and (ii) phenylalkyl siloxysilicate resins, the alkyl of the phenylalkyl is as defined above, such as phenylpropyldimethyl siloxysilicate.

Examples of such MQ type silicone resins also include, but are not limited to, trimethyl siloxysilicate type, such as those sold under the reference SR1000 by the company General Electric, under the reference TMS 803 by the company Wacker, or under the name KF-7312J by the company Shin-Etsu or DC 749 or DC 593 by the company Dow Corning.

Examples of such MQ type silicone resins further include, but are not limited to, MQ siloxysilicate units, such as phenylalkylsiloxysilicate resins like phenylpropyldimethylsiloxysilicate (Silshine 151 sold by the company General Electric).

### Fluid Silicone Segment

According to one embodiment of the present invention, the fluid silicone segment of the copolymer according to the present invention bears an OH end function group.

Preferably, the fluid silicone segment is a diorganopolysiloxane bearing OH end functions, having a viscosity of between 100 and 100,000 cSt at 25° C. (determined with Brookfield viscometer using ASTMD-445 method), for which the substituent(s) of the diorganopolysiloxane are independently chosen from methyl, ethyl, propyl and vinyl radicals. The diorganopolysiloxanes are preferably linear polymers. Examples of diorganopolysiloxanes may be, in a non-limiting manner, a polydimethylsiloxane, an ethylmethyl polysiloxane, a copolymer of dimethylsiloxane and of methylvinylsiloxane, and mixtures of such polymers or copolymers containing OH end groups. The preferred diorganopolysiloxane is a polydimethylsiloxane.

For example, the copolymers according to the present invention may be prepared by heating the following mixture of:
from 45% to 75% by mass of silicone resin, being the product of condensation of SiO 2 and R 3 (SiO) ½ units for which each group R is independently selected from methyl, ethyl, propyl and vinyl radicals and for which the ratio between the SiO 2 functions and the R 3 (SiO) ½ functions of the silicone resin ranges from 0.6 to 0.9;
from 25% to 55% by mass of fluid diorganopolysiloxane containing OH end functions, with a viscosity of between 100 and 100,000 cSt at 25° C. (determined with Brookfield viscometer using ASTMD-445 method), for which the substituents of the diorganopolysiloxane are independently chosen from methyl, ethyl, propyl and vinyl radicals; and
from 0.001% to 5% of a suitable catalyst, which is preferably an organic aliphatic amine compound preferably chosen from primary amines, secondary amines, tertiary amines, carboxylic acid salts of the amines mentioned above and quaternary ammonium salts.

The mixture is heated to a temperature of between 80° C. and 160° C. until the adhesive nature of the resulting silicone copolymer is obtained.

In the copolymer, the silicone resin is present in a concentration by weight ranging from about 0.5%, 1%, 2.5% or 5% to about 15%, 17.5%, 20% or 25% by weight, including all ranges and subranges therebetween.

Examples of the copolymers containing a silicone resin segment and a fluid silicone segment that are notable according to the present invention are sold by Dow Corning under the reference Bio-PSA® and DOWSIL™. Mention may be made especially of the grades 7-4400, 7-4405, 7-4500 and 7-4600 for Bio-PSA® and FC-5001 CM Resin Gum, FC-5002 IDD Resin Gum and FC-5004 DM Resin Gum for DOWSIL™. The copolymer containing a silicone resin segment and a fluid silicone segment may be described by the INCI name Trimethylsiloxysiliate/Dimethiconol Crosspolymer (also referred to as “TMSS/DC” herein).

Advantageously, the copolymer containing a silicone resin segment and a fluid silicone segment is present in an amount ranging from 0.5%, 1% or 2% to 10%, 15% or 25%, preferably from 0.8% to 8%, more preferably from 1% to 6%, relative to the weight of the composition.

### Hydrophobically-Modified Pullulan Compound Modified

According to the present invention, compositions including one or more hydrophobically-modified pullulan compound(s) are provided.

Pullulan is a natural polysaccharide produced from starch by the fungus Aureobasidium pullulans. Pullulan is an alpha-glucan mainly constituted of maltotriose repeating units linearly joined through alpha 1,6-glycosidic linkages. Natural pullulan is readily soluble in cold or warm water and forms clear, viscous solutions in it.

Pullulan may be produced, for example, from the fermentation of partially hydrolyzed starch or by using sucrose, sugar cane, milk, potato and other sources for carbohydrate starting materials.

Preferably, hydrophobically-modified pullulan compounds of the present invention are oil-soluble. For example, preferred pullulan compounds of the present invention have been modified with a hydrophobic group such as a siloxane group or a hydrocarbon group which imparts oil-solubility to the compound. Such modifications can include, for example, a silicone group such as a siloxysilyl group (—[—SiRR′—SiOR″R′″—]—), where R, R′, R″ and R′″ are alkyl groups preferably containing 1 to 8 carbon atoms, preferably 1 to 4 carbon atoms and preferably 1 to 3 carbon atoms), or the can include C8 or greater hydrocarbon groups (for example, hydrocarbon groups containing 8 to 24 carbon atoms including ranges and subranges therebetween such as 12 to 22 carbon atoms and 12 to 18 carbon atoms).

A preferred siloxane-modified pullulan compound has a structure corresponding to:
pullulan-linker-siloxane group;
where the siloxane group is preferably a C1-C6 alkyl-substituted siloxysilyl group such as, for example, a monoalkyl-substituted siloxysilyl group, a diakyl-substituted siloxysilyl group, or a trialkyl-substituted siloxysilyl group, where the C1-C6 alkyl group is linear, cyclic or branched, substituted or unsubstituted; and
where the linker does not adversely affect either the film forming properties or the oil-solubility properties of the compound. Preferably, the linker is alkyl (—C—), ester or ether (—O—), amide or amine (—N—) or a combination thereof such as a carbamoyl group. Preferably, the linker is a carbamoyl group.

Preferably, the hydrophobically-modified pullulan compound is a trialkylsiloxysilylcarbamoyl pullulan, with a particularly preferred compound being trimethylsiloxysilylcarbamoyl pullulan. The hydrophobically-modified pullulan compound may be described by the INCI name Trimethylsiloxysilylcarbamoyl Pullulan (also referred to as “TMSSCP” herein).

Preferably, hydrophobically-modified pullulan compound(s) in the compositions of the present invention have a molecular weight of 50,000 to 10,000,000 daltons, preferably 100,000 to 5,000,000 daltons, preferably 250,000 to 1,000,000 daltons, including all ranges and subranges therebetween including, for example, 200,000 to 7,600,000 daltons, 350,000 to 8,750,000 daltons, 225,000 to 700,000 daltons, etc.

Preferably, the at least one hydrophobically-modified pullulan compound is present in the compositions of the present invention in an amount ranging from about 0.5%, 1%, 2.5% or 5% to about 5%, 15%, 17.5%, 20% or 25% by weight, including all ranges and subranges therebetween.

The inventors have further found that the ratio of concentrations by weight of the (A) at least one copolymer containing a silicone resin segment and at least one fluid silicone segment and the (B) at least one hydrophobically-modified pullulan compound, A:B is important indicator of performance. In certain embodiments the ratio of A:B is from about 1:10 or 1:4 to about 4:1 or 10:1. According to certain other embodiments, the ratio A:B is greater than 1:1, such as greater than about 2:1, such as greater than about 3:1. This ratio of (A):(B) may be, for example greater than 1:1 or from about 1.25:1, 1.5:1, 2:1 or 3:1 to about 3:1, 4:1, 5:1, 6:1, 7:1, 8:1, 9:1 or 10:1, such as, for example, greater than 1:1 to about 10:1, such as from about 2:1 or 3:1 to about 4:1, 5:1. 6:1 or 10:1.

According to certain embodiments, the film forming polymers are predominantly comprised of the (1) at least one copolymer containing a silicone resin segment and at least one fluid silicone segment and (2) at least one hydrophobically-modified pullulan compound. In this embodiment at least 50%, such as at least 75%, such as at least 90% by weight of all film-forming polymers in the composition are from the aforementioned polymers.

According to other embodiments the sum total concentration by weight of the (1) at least one copolymer containing a silicone resin segment and at least one fluid silicone segment and (2) at least one hydrophobically-modified pullulan compound is from about 0.5%, 2%, 5%, 7.5%, or 10% to about 15%, 17.5%, 20%, 22.5%, 25% or 30%.

### Oil Phase

According to preferred embodiments of the present invention, compositions further comprising at least one fatty substance are provided. Suitable fatty substances include oil(s) and/or wax(es). “Oil” means any non-aqueous medium which is liquid at ambient temperature (25° C.) and atmospheric pressure (760 mm Hg). A “wax” for the purposes of the present disclosure is a lipophilic fatty compound that is solid at ambient temperature (25° C.) and changes from the solid to the liquid state reversibly, having a melting temperature of more than 30° C. and, for example, more than 45° C., which can be as high as 150° C., a hardness of more than 0.5 MPa at ambient temperature, and an anisotropic crystalline organization in the solid state. By taking the wax to its melting temperature, it is possible to use wax(es) by themselves as carriers and/or it is possible to make wax(es) miscible with the oils to form a microscopically homogeneous mixture.

Suitable oils include volatile and/or non-volatile oils. Such oils can be any acceptable oil including but not limited to silicone oils and/or hydrocarbon oils.

According to certain embodiments, the compositions of the present invention preferably comprise one or more volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |

Further, a volatile linear silicone oil may be employed in the present invention. Suitable volatile linear silicone oils include those described in U.S. Pat. No. 6,338,839 and WO 03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to certain embodiments of the present invention, the composition preferably comprises one or more non-silicone volatile oils and may be selected from volatile hydrocarbon oils, volatile esters and volatile ethers such as those discussed above. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins) or C₉ to C₁₂ alkanes, isohexacecane, isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |
| Undecane | 62 |
| Tridecane | 94 |
| Isohexadecane | 96 |

The volatility of the solvents/oils can be determined using the evaporation speed as set forth in U.S. Pat. No. 6,338,839, the contents of which are incorporated by reference herein. According to preferred embodiments, if present, the at least one volatile oil is present in the compositions of the present invention in an amount ranging from about 10%, 15%, 20%, 25% or 30% to about 30%, 35%, 40%, 50%, 60% or 80% by weight, including all ranges and subranges within these ranges. In certain other embodiments, the concentration of volatile, non-silicone oil falls within one or more of these specific ranges as well.

According to certain embodiments of the present invention, the composition comprises at least one non-volatile oil. Examples of non-volatile oils that may be used in the present invention include, but are not limited to, polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
synthetic oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, with R₆+R₇≥10, such as, for example, Purcellin oil (cetostearyl octanoate), isononyl isononanoate, octyldodecyl neopentanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
synthetic ethers containing from 10 to 40 carbon atoms;
C₈ to C₂₆ fatty alcohols, for instance oleyl alcohol, cetyl alcohol, stearyl alcohol, and cetearyl alcohol; and
mixtures thereof.

Further, examples of non-volatile oils that may be used in the present invention include, but are not limited to, non-polar oils such as branched and unbranched hydrocarbons and hydrocarbon waxes including polyolefins, in particular Vaseline (petrolatum), paraffin oil, squalane, squalene, hydrogenated polyisobutene, hydrogenated polydecene, polybutene, mineral oil, pentahydrosqualene, and mixtures thereof.

Further, examples of non-volatile oils that may be used in the present invention include, but are not limited to, silicone oils such as dimethicone (polydimethylsiloxane) of various viscosities as well as phenylated silicone oils such as phenyl trimethicone and trimethylsiloxyphenyl dimethicone.

According to preferred embodiments, if present, the at least one oil is present in the compositions of the present invention in an amount ranging from about 10%, 15%, 20%, 25% or 30% to about 60%, 65%, 70%, or 80% by weight, including all ranges and subranges within these ranges.

According to preferred embodiments of the present invention, the compositions of the present invention further comprise at least one wax. Suitable examples of waxes that can be used in accordance with the present disclosure include those generally used in the cosmetics field: they include those of natural origin, such as beeswax, carnauba wax, candelilla wax, ouricoury wax, Japan wax, cork fibre wax or sugar cane wax, rice bran wax, rice wax, montan wax, paraffin wax, lignite wax or microcrystalline wax, ceresin or ozokerite, and hydrogenated oils such as hydrogenated castor oil or jojoba oil; synthetic waxes such as the polyethylene waxes obtained from the polymerization or copolymerization of ethylene, and Fischer-Tropsch waxes, or else esters of fatty acids, such as octacosanyl stearate, glycerides which are solid at 30° C., for example at 45° C.

According to certain embodiments of the present invention, the compositions of the present invention may include at least one silicone wax. Examples of suitable silicone waxes include, but are not limited to, silicone waxes such as alkyl- or alkoxydimethicones having an alkyl or alkoxy chain ranging from 10 to 45 carbon atoms, poly(di)methylsiloxane esters which are solid at 30° C. and whose ester chain comprising at least 10 carbon atoms, di(1,1,1-trimethylolpropane) tetrastearate, which is sold or manufactured by Heterene under the name HEST 2T-4S; alkylated silicone acrylate copolymer waxes comprising at least 40 mole % of siloxy units having the formula (R₂R′SiO_{1/2})ₓ(R″SiO_{3/2})_{y}, where x and y have a value of 0.05 to 0.95, R is an alkyl group having from 1 to 8 carbon atoms, an aryl group, a carbinol group, or an amino group, R is a monovalent hydrocarbon having 9-40 carbon atoms, R″ is a monovalent hydrocarbon group having 1 to 8 carbon atoms, an aryl group such as those disclosed in U.S. Patent Application No.: 2007/0149703, the entire contents of which is hereby incorporated by reference, with a particular example being C30-C45 alkyldimethylsilyl polypropylsilsesquioxane; and mixtures thereof.

According to preferred embodiments, the compositions of the present invention contain less than 1% wax.

According to preferred embodiments, the compositions of the present invention contain less than 0.5% wax.

According to preferred embodiments, the compositions of the present invention contain no wax.

If present, the wax or waxes may be present in an amount ranging from 1 to 30% by weight relative to the total weight of the composition, for example from 2 to 20%, and for example from 3 to 10%, including all ranges and subranges therebetween.

### Coloring Agents

According to preferred embodiments of the present invention, compositions of the present invention may optionally further comprise at least one coloring agent. According to this embodiment, the coloring agent(s) is/are preferably chosen from pigments, dyes, nacreous pigments, pearling agents, and mixtures thereof.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 40% by weight of the total weight of the composition, such as from 0.0001% to 30%, including all ranges and subranges therebetween.

The nacreous pigments which may be used according to the present invention may be chosen from colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, be present in the composition in a concentration ranging up to 50% by weight of the total weight of the composition, such as from 0.0001% to 40%, preferably from 0.001% to 30%, including all ranges and subranges therebetween.

The pigments, which may be used according to the present invention, may be chosen from white, colored, inorganic, organic, polymeric, and nonpolymeric pigments. Representative examples of mineral pigments include titanium dioxide, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the coloring agents may be present in the composition in a concentration ranging up to 50% by weight of the total weight of the composition, such as from 0.0001% to 40%, and further such as from 0.001% to 30%, including all ranges and subranges therebetween.

### Aqueous Phase

The compositions of the present invention may also optionally contain water. When the compositions of the present invention contain water, they are preferably in the form of an emulsion. Preferably, when the compositions of the present invention contain water, they are in the form of an oil-in-water emulsion (O/W) or a water-in-oil emulsion (W/O). Preferably, when in the form of an emulsion, the oil phase contains predominantly silicone oils (Si/W or W/Si emulsion) or hydrocarbon oils. When present, water is preferably present in an amount of from about 10% to about 80% by weight, preferably from about 20% to about 70% by weight, preferably from about 35% to about 65% by weight, including all ranges and subranges therebetween, all weights being based on the total weight of the composition.

According to preferred embodiments, however, compositions of the present invention are devoid of water, substantially free of water, or free of water as defined herein. Preferably, the compositions of the present invention are anhydrous.

The composition of the invention can also comprise any additive usually used in the field under consideration. For example, additional film formers other than the at least one and the at least one (1) at least one copolymer containing a silicone resin segment and at least one fluid silicone segment and (2) at least one hydrophobically-modified pullulan compound, dispersants such as poly(12-hydroxystearic acid), antioxidants, essential oils, sunscreens, preserving agents, fragrances, fillers, neutralizing agents, cosmetic and dermatological active agents such as, for example, emollients, moisturizers, vitamins, essential fatty acids, surfactants, silicone elastomers, thickening agents, gelling agents, particles, pasty compounds, viscosity increasing agents can be added. A non-exhaustive listing of such ingredients can be found in U.S. Patent Application Publication No.: 2004/0170586, the entire contents of which is hereby incorporated by reference. Further examples of suitable additional components can be found in the other references which have been incorporated by reference in this application.

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the composition of the invention should be cosmetically or dermatologically acceptable, i.e., it should contain a non-toxic physiologically acceptable medium and should be able to be applied to the keratinous materials of human beings such as, for example, lips, skin or eyelashes.

In particular, suitable gelling agents for the oil phase include, but are not limited to, lipophilic or hydrophilic clays.

The term “hydrophilic clay” means a clay that is capable of swelling in water; this clay swells in water and forms after hydration a colloidal dispersion. Clays are silicates containing a cation that may be chosen from calcium, magnesium, aluminium, sodium, potassium and lithium cations, and mixtures thereof. Examples of such products that may be mentioned include clays of the smectite family such as montmorillonites, hectorites, bentonites, beidellites and saponites, and also of the family of vermiculites, stevensite and chlorites. These clays may be of natural or synthetic origin.

Hydrophilic clays that may be mentioned include synthetic hectorites (also known as laponites), for instance the products sold by the company Laporte under the names Laponite XLG, Laponite RD and Laponite RDS (these products are sodium magnesium silicates and in particular sodium lithium magnesium silicates); bentonites, for instance the product sold under the name Bentone HC by the company Rheox; magnesium aluminium silicates, especially hydrated, for instance the products sold by the Vanderbilt Company under the names Veegum Ultra, Veegum HS and Veegum DGT, or calcium silicates, and especially the product in synthetic form sold by the company under the name Micro-cel C.

The term “lipophilic clay” means a clay that is capable of swelling in a lipophilic medium; this clay swells in the medium and thus forms a colloidal dispersion. Examples of lipophilic clays that may be mentioned include modified clays such as modified magnesium silicate (Bentone Gel VS38 from Rheox), and hectorites modified with a C₁₀ to C₂₂ fatty-acid ammonium chloride, for instance hectorite modified with distearyldimethylammonium chloride (CTFA name: disteardimonium hectorite) sold under the name Bentone 38 CE by the company Rheox or Bentone 38V® by the company Elementis.

In particular, among the gelling agents that may be used, mention may be made of silica particles. Preferably, the silica particles are fumed silica particles.

Suitable silicas include, but are not limited to, hydrophobic silicas, such as pyrogenic silica optionally with hydrophobic surface treatment whose particle size is less than 1 micron, preferably less than 500 nm, preferably less than 100 nm, preferably from 5 nm to 30 nm, including all ranges and subranges therebetween. It is in fact possible to modify the surface of silica chemically, by a chemical reaction producing a decrease in the number of silanol groups present on the surface of the silica. The silanol groups can notably be replaced with hydrophobic groups: a hydrophobic silica is then obtained. The hydrophobic groups can be:

trimethylsiloxyl groups, which are notably obtained by treatment of pyrogenic silica in the presence of hexamethyldisilazane. Silicas treated in this way are called “Silica silylate” according to the CTFA (6th edition, 1995). They are for example marketed under the references “AEROSIL R812®” by the company Degussa, “CAB-O-SIL TS-530®” by the company Cabot;

dimethylsilyloxyl or polydimethylsiloxane groups, which are notably obtained by treatment of pyrogenic silica in the presence of polydimethylsiloxane or dimethyldichlorosilane. Silicas treated in this way are called “Silica dimethyl silylate” according to the CTFA (6th edition, 1995). They are for example marketed under the references “AEROSIL R972®”, “AEROSIL R974®” by the company Degussa, “CAB-O-SIL TS-610®”, “CAB-O-SIL TS-720®” by the company Cabot.

Preferably, the gelling agent, if present, is present in the composition of the present invention in amounts of active material generally ranging from about 0.1% to about 10%, preferably from about 0.25% to about 5%, and more preferably from about 0.5% to about 3.5%, by weight, based on the total weight of the composition, including all ranges and subranges in between.

According to preferred embodiments of the present invention, methods of treating, caring for and/or making up a keratinous material by applying compositions of the present invention to the keratinous material in an amount sufficient to treat, care for and/or make up the keratinous material are provided. Preferably, “making up” the keratinous material includes applying at least one coloring agent to the keratinous material in an amount sufficient to provide color to the keratinous material.

According to yet other preferred embodiments, methods of enhancing the appearance of a keratinous material by applying compositions of the present invention to the keratinous material in an amount sufficient to enhance the appearance of the keratinous material are provided.

In accordance with the preceding preferred embodiments, the compositions of the present invention are applied topically to the desired area of the keratinous material in an amount sufficient to treat, care for and/or make up the keratinous material, to cover or hide defects associated with keratinous material, or to enhance the appearance of keratinous material. The compositions may be applied to the desired area as needed, preferably once daily, and then preferably allowed to dry before subjecting to contact such as with clothing or other objects. Preferably, the composition is allowed to dry for about 4 minutes or less, more preferably for about 2 minutes or less.

In preferred embodiments, methods of making up the lips are provided, wherein the method includes applying compositions provided herein to the users lips in an amount sufficient to make up the lips.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### Example 1—Sample Invention Compositions

The following compositions can be prepared:

| | General Range | Specific Range |
|---|---|---|
| Ingredient | (active content) | (active content) |
| Copolymer Containing A | 0.5%-25% | 2%-15% |
| Silicone Resin Segment | | |
| And A Fluid Silicone | | |
| Segment (e.g., TMSS/DC) | | |
| Hydrophobically-modified | 0.5%-25% | 0.5%-5% |
| pullulan compound | | |
| (e.g., TMSSCP) | | |
| Volatile Oil | 10%-80% | 25%-60% |
| Coloring Agent | 0.1%-30% | 5%-20% |
| Other (e.g., preservatives, | 0%-10% | 0.1%-5% |
| wax, active agents, etc.) | | |

### Example 2—Testing Procedures

### Example 2A: Extended Wear Assay Test Procedure

A 1 mL drawdown of samples (below) was performed on abrasion paper, and the drawdowns were allowed to sit at ambient temperature for 24 hours to dry and form a film. Two droplets of liquid (olive oil, artificial saliva or acetic acid) were placed on the film of each sample tested, making sure not to overlap the droplets. The droplets were allowed to sit for the indicated times below and then were wiped with a cotton round (pad) 15 times. This procedure was repeated twice to produce an average score.

Let the 1 mL draw down of test composition on abrasion paper sit on bench top at ambient temperature for 24 hours to dry

Use on piece of ASTM tape to traverse the right side of the film, with slight finger pressure, run across the film and at a 180 degree angle, pull pack the film gently to remove

On the draw down place droplets on the draw down film. 2 droplets of olive oil and repeated for artificial saliva and acetic acid making sure not to overlap the droplets.

Let droplets sit for 10 min and then wipe with cotton round 15 times for each category.

The above procedure was repeated for each sample for each liquid (olive oil, artificial saliva, acetic acid).

Finally, to a film on which no liquid has been applied, run a piece of ASTM tape on the film, with slight finger pressure and removed at a 180 degree angle.

Grading: The cotton round and the substrate are viewed and graded as to how much product is remaining. Numerical rating on a scale of 1-3 was assigned where 1=least transfer/best film property and 3=most transfer/worst film property. An average score of two readings was obtained and recorded in the table below—where “Oil” refers to the substrate for the oil challenge; “Oil KW” refers to the cotton round for the oil challenge; “Saliva” refers to the substrate for the saliva challenge; “Saliva KW” refers to the cotton round for the saliva challenge; “Acetic acid” refers to the substrate for the acetic acid challenge; “A.A. wipe” refers to the cotton round for the acetic acid challenge. The average of scores is also reported.

### Example 2B: Stretch Test Procedure

Each Sample (below) was drawn down on 3×1 window on a 4×2 section of nitrile glove with a ½″ border on all sides created using tape, and each sample was allowed to dry overnight and the tape was removed. Then, each sample was stretch 10 times a total of 6 inches to determine the impact on flexibility, using the following grading scale:

Rating Scale

| | |
|---|---|
| 0 | No breakage |
| 1 | Minimum breakage |
| 2 | Some breakage |
| 3 | Half breakage |
| 4 | Significant breakage |
| 5 | Total breakage |

### Example 3: Testing

Compositions were prepared as follows:

| Ingredient | Ex 1 | Ex 2 | Ex3 | C1 | C2 |
|---|---|---|---|---|---|
| Trimethylsiloxysilicate/ | 9.6 | 6.0 | 2.4 | 12.0 | 0 |
| Dimethiconol | | | | | |
| Crosspolymer (TMSS/DC) | | | | | |
| Trimethylsiloxysilylcarbamoyl | 2.4 | 6.0 | 9.6 | 0 | 12.0 |
| Pullulan (TMSSCP) | | | | | |
| Acetyl Tributyl Citrate | 2.0 | 2.0 | 2.0 | 2.0 | 2.0 |
| Disteardimonium | 4.0 | 4.0 | 4.0 | 4.0 | 4.0 |
| hectorite and Propylene | | | | | |
| carbonate | | | | | |
| Pigment | 10.0 | 10.0 | 10.0 | 10.0 | 10.0 |
| Isododecane | Q.S. | Q.S | Q.S | Q.S | Q.S |
| Total TMSS/DC + TMSSCP | 12 | 12 | 12 | 12 | 12 |
| Ratio(A:B) | 4:1 | 1:1 | 1:4 | — | — |
| TMSS/DC:TMSSCP | | | | | |

These compositions were tested for extended wear and flexibility according to the procedures in Example 2, where the droplets were allowed to sit for 10 minutes. The results of this testing are set forth below:

| Sam- | | Oil | | Saliva | Acetic | A.A. | Ave. | |
|---|---|---|---|---|---|---|---|---|
| ple | Oil | KW | Saliva | KW | Acid | Wipe | Score | Stretch |
| Ex 1 | 1.25 | 2.00 | 1.00 | 1.25 | 1.00 | 1.00 | 1.25 | 0.5 |
| Ex 2 | 1.00 | 1.63 | 1.00 | 1.25 | 1.00 | 1.25 | 1.19 | 1.0 |
| Ex 3 | 1.00 | 1.50 | 1.00 | 1.25 | 1.00 | 1.50 | 1.21 | 2.0 |
| C1 | 1.75 | 2.00 | 1.00 | 1.00 | 1.00 | 1.00 | 1.29 | 0.5 |
| C2 | 1.80 | 1.50 | 1.00 | 1.25 | 1.00 | 1.50 | 1.27 | 4.0 |

In comparing Examples Ex1, Ex 2 and Ex3 with C1 and C2, it can be seen that surprisingly all of the inventive compositions provide better average wear scores (and dramatically so for the “oil” challenge) than either polymer (A) or (B) alone. Further, it can also be seen that the (A:B) ratio of 4:1 and not the 1:1 or 1:4 ratios surprisingly also maintain the excellent stretch properties that are present in the “all-TMSS/DC” system (C1).

What is claimed is:

1. A composition comprising (A) at least one copolymer containing a silicone resin segment and at least one fluid silicone segment and (B) at least one hydrophobically-modified pullulan compound.

2. The composition of claim 1, wherein the (A) at least one copolymer containing a silicone resin segment and at least one fluid silicone segment and the (B) at least one hydrophobically-modified pullulan compound are present in a ratio by weight, A:B, that is greater than 1:1.

3. The composition of claim 1, wherein the (A) at least one copolymer containing a silicone resin segment and at least one fluid silicone segment and the (B) at least one hydrophobically-modified pullulan compound are present in a ratio by weight, A:B, that is at least about 2:1.

4. The composition of claim 1, wherein the (A) at least one copolymer containing a silicone resin segment and at least one fluid silicone segment and the (B) at least one hydrophobically-modified pullulan compound collectively represent a majority fraction of a film-forming component of the composition.

5. The composition of claim 1, wherein the composition is anhydrous.

6. The composition of claim 1, wherein the composition is liquid.

7. The composition of claim 1, wherein the composition further comprises at least one coloring agent.

8. The composition of claim 1, wherein the composition further comprises at least one volatile oil.

9. The composition of claim 1, wherein the composition further comprises from about 10% to about 60% by weight of at least one volatile oil and wherein the (A) at least one copolymer containing a silicone resin segment and at least one fluid silicone segment and (B) at least one hydrophobically-modified pullulan compound are present in a total percent by weight from about 2% to about 20%, wherein all percentages are of the total composition.

10. The composition of claim 1, wherein (A) at least one copolymer containing a silicone resin segment and at least one fluid silicone segment and the (B) at least one hydrophobically-modified pullulan compound are present in a ratio by weight, A:B, that is greater than 1:1 to about 10:1.

11. A method of making up lips comprising applying the composition of claim 1 to lips in an amount sufficient to makeup the lips.

12. The method of claim 11, wherein the (A) at least one copolymer containing a silicone resin segment and at least one fluid silicone segment and the (B) at least one hydrophobically-modified pullulan compound are present in a ratio by weight, A:B, that is greater than 1:1.

13. The method of claim 11, wherein the (A) at least one copolymer containing a silicone resin segment and at least one fluid silicone segment and the (B) at least one hydrophobically-modified pullulan compound are present in a ratio by weight, A:B, that is greater than about 2:1.

14. The method of claim 11, wherein the (A) at least one copolymer containing a silicone resin segment and at least one fluid silicone segment and the (B) at least one hydrophobically-modified pullulan compound collectively represent a majority fraction of a film-forming component of the composition.

15. The method of claim 11, wherein the composition is anhydrous.

16. The method of claim 11, wherein the composition is liquid.

17. The method of claim 11, wherein the composition further comprises at least one coloring agent.

18. The method of claim 11, wherein the composition further comprises at least one volatile oil.

19. The method of claim 11, wherein the composition further comprises from about 10% to about 60% by weight of at least one volatile oil and wherein the (A) at least one copolymer containing a silicone resin segment and at least one fluid silicone segment and (B) at least one hydrophobically-modified pullulan compound are present in a total percent by weight from about 2% to about 20%, wherein all percentages are of the total composition.

20. The method of claim 11, wherein (A) at least one copolymer containing a silicone resin segment and at least one fluid silicone segment and the (B) at least one hydrophobically-modified pullulan compound are present in a ratio by weight, A:B, that is greater than 1:1 to about 10:1.
